(19) United States
(12) Patent Application Publication
(10) Pub. No.: US 2006/0241665 A1
(43) Pub. Date: Oct. 26, 2006

(54) Percutaneous and endoscopic cutters

(75) Inventors: Rodney W. Bosley, Chester Springs, PA (US); Tracy E. Willis, Bloomington, IN (US); Geoffrey P. Grant, Bloomington, IN (US)

Correspondence Address: BRINKS HOFER GILSON & LIONE/CHICAGO/COOK, PO BOX 10395, CHICAGO, IL, 60610, US

(73) Assignees: Vance Products Incorporated, d/b/a Cook Urological Incorporated, Spencer, IN (US); Sabin Corporation, a Cook Group Company, Bloomington, IN (US)

(21) Appl. No.: 11/399,781

(22) Filed: Apr. 7, 2006

Related U.S. Application Data
(60) Provisional application No. 60/669,446, filed on Apr. 8, 2005.

(51) Int. Cl. A61B 17/32

(52) U.S. Cl. 606/167

(57) ABSTRACT

A surgical cutter with a flat blade and a cutting wedge allows a surgeon to safely trim a foreign object inside a body, such as a urethral sling inside a patient, so that a minimum of sling remains in place after cutting. The shortened sling minimizes tension applied to the urethra and to any nearby bladder tissues. The cutter may also be used to trim other prostheses or devices implanted within a patient. The cutter is configured so that the cutting surface of the blade does not contact tissue of the patient.

This application claims the benefit of the filing date under 35 U.S.C. § 119(e) of Provisional U.S. patent application Ser. No. 60/669,446, filed on Apr. 8, 2005, which is hereby incorporated by reference in its entirety.

### FIELD OF THE INVENTION

The field of the invention is that of surgical instruments.

### BACKGROUND

Surgical procedures often involve placing a device or prosthesis inside a person, the prosthesis substituting for a missing or damaged organ or body part. The prosthesis is intended to at least partially replace the natural part, and to assume the functions of the natural part. In recent years, medical procedures have advanced to the point where many procedures, previously believed to be difficult and rare, and now much more commonplace.

Included in these procedures is the placement of a support sling under the urethra, primarily in women, to alleviate urinary stress incontinence. Women who have given birth several times may be prone to this disorder, which is caused by the general relaxation of the pelvic floor, and its failure to properly support the urethra. If the support structure for the urethra is poor, the attitude of the urethra may change during momentary stress, such as coughing, sneezing, or laughing. The urethra may change its attitude in a downward manner, and leakage from the bladder may occur.

This is an embarrassing problem, for which a number of remedies have been proposed, primarily since 1942, when Dr. Aldridge published one of the first works in the field, Transplantation of Fascia for Relief of Urinary Stress Incontinence, Vol. 44, American J. Obstetrics and Gynecology, p. 398 (1942). Since that time, a number of materials, procedures, and techniques have been devised, most of them involving the placement of a sling, such as a sling made from fascia lata, or other materials. The sling is typically placed under the urethra, or under the urethra and the neck of the bladder, to support the urethra and prevent a change in the angle of the urethra during momentary stress.

One problem has been the placement of the sling in a manner calculated to prevent tension on the sling during normal activity of the wearer. Tension on the sling may lead to unwanted stress on the urethra or even on the bladder, if placement is too close to the bladder. As a result, physicians place the slings in a manner so that the slings are not loose, nor tight, but so that the slings will support the urethra during momentary stress and without tension during normal activity.

One way to alleviate any potential problem of stress is to shorten the length of sling placed under the urethra. If the sling were shorter, it would be less able to support itself with tension than a longer sling. Accordingly, it would be beneficial if a physician is able to place a shorter sling.

### BRIEF SUMMARY

One aspect of the invention is a cutter for cutting an object inside a body, the cutter including a housing having a longitudinal axis, a plunger mounted within the housing for longitudinal, manual actuation, a distal end of the plunger mounting one of a flat blade and a wedge, and a distal end of the housing fixedly mounting the other of the flat blade and the wedge, and an opening in the housing between the flat blade and the wedge, the opening configured to admit an object for cutting and not to admit a part of a body, and wherein the blade is contained within the cutter.

Another aspect of the invention is a cutter for cutting an object inside a body, the cutter including a rigid housing, a handle operably mounted to the housing, a plunger mounted inside the housing and mounting one of a flat blade and a wedge, the plunger configured for longitudinal, manual actuation, wherein the other of the flat blade and the wedge is fixedly mounted to the housing, and a first slot in the housing oriented at an angle to the housing.

Another aspect of the invention is a method of cutting an object inside a body. The method includes placing the cutter into the body of a patient, wherein the cutter comprises a housing having a longitudinal axis and a distal opening, a plunger mounted within the housing for longitudinal, manual actuation and mounting one of a flat blade and a wedge, and a distal portion of the housing fixedly mounting the other of the flat blade and the wedge. The method also includes drawing the object into the opening of the cutter, pushing on a portion of the cutter to bring a cutting surface of the flat blade into contact with the object to cut the object, and removing the object which was cut.

There are many aspects of this invention, only a few of which are shown in the appended figures and in the attached description of the presently preferred embodiments.

### BRIEF DESCRIPTION OF THE DRAWINGS

FIG. 1 is an isometric view of a first embodiment of a cutter;

FIGS. 1a and 1b are isometric views of top and bottom halves of the embodiment of FIG. 1

FIG. 2 is an isometric view of a blade assembly or plunger useful in the embodiment of FIG. 1;

FIG. 3 is a view of a blade stop useful in embodiments of the invention;

FIG. 4 is a partial cutaway view of another embodiment;

FIG. 5 is an exploded view of another embodiment of a blade assembly;

FIG. 6 is an alternative embodiment of a distal portion of the housing;

FIG. 7 is a partially broken-away isometric view of another embodiment;

FIG. 8 is another embodiment of a plunger and a blade;

FIGS. 9-10 depict an endoscope and flexible cutter used therewith; and

FIGS. 11-14 are handles useful for endoscopic or percutaneous cutters.

### DETAILED DESCRIPTION OF THE DRAWINGS AND THE PRESENTLY PREFERRED EMBODIMENTS

Embodiments of the present invention are intended for use within a human or animal body, although they are not limited to use within a body. The cutting device should therefore be sterile, and is preferably intended for use with only one patient. In some embodiments, the device is rigid and is intended to penetrate a limited distance into the body, using a path already created. In other embodiments, the cutter is flexible and may be inserted through an endoscope or other device for minimally-invasive surgery. The cutter is preferably thin and as narrow as possible, consistent with the width and thickness of the sling or other device it is intended to cut. For instance, in order to cut a urethral sling that is about 1 cm wide, the cutter preferably but not necessarily, should also be about 1 cm wide, so that the sling may present a neat, flat surface for cutting. The cutter may alternatively be narrower or wider than the sling or other part to be cut or trimmed.

The cutter may be used as described below, for trimming a device or prosthesis that has been implanted within the body. The advantage of implanting within the body is that the surgeon or other medical professional is able to work with the uncut, full length of the device before cutting. For instance, urethral slings are much more easily placed before cutting, since they may be drawn using their full length from the area of the urethra to the desired area for suspension, and then trimmed to the desired length. The sling could be cut beforehand and drawn into place using a suture. However, the surgeon would have to estimate the desired length before implantation. In addition, with the cut made beforehand, the surgeon would be able to work only with the suture for placement, rather than an additional length of the sling. The additional length of the sling before cutting makes placement much easier. Using only a suture, rather than the sling itself, would make the procedure, and precise placement of the sling, more difficult.

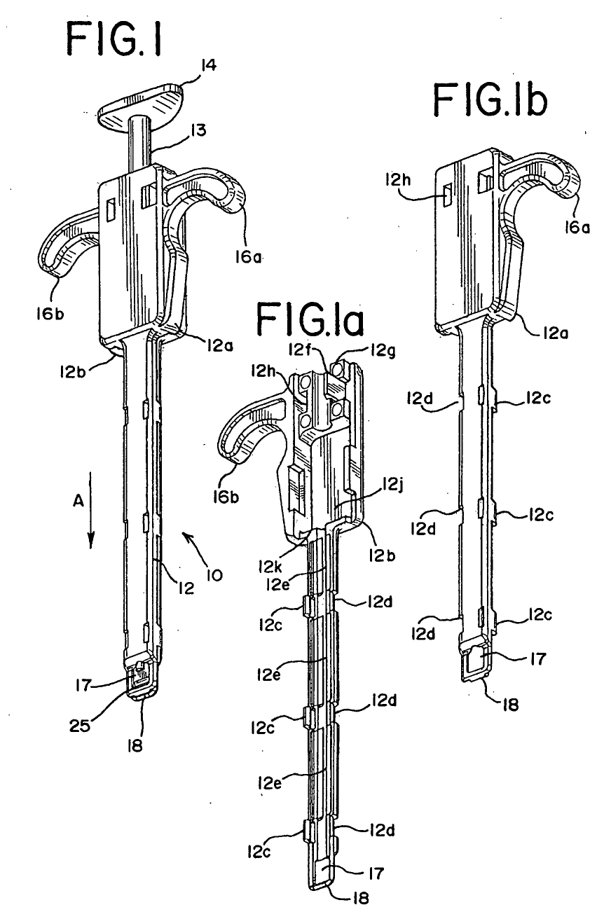

FIG. 1 depicts a first embodiment of a cutter 10 for trimming the length of a urethral sling. The cutter includes a rigid housing 12 made of a top half 12a and a bottom half 12b. Top half 12a includes a left handle 16a and bottom half 12b includes a right handle 16b. The cutter also includes a control rod or plunger 13 with an actuation surface 14 at a proximal portion of the plunger, and also including a blade 25 at a distal portion of the plunger. The cutter includes an opening or space 17 through which the material to cut is drawn, and a distal portion 18, acting as a pinch point for the cutting surface of the blade 25.

In use, a physician or other medical professional draws a sling, preferably using a suture, though the opening, and then depresses the patient's abdomen while gently restraining the sling and pushing the cutter into the body pathway. When the desired degree of penetration is achieved, and the desired amount of sling has passed through the opening, the physician actuates the cutter by depressing the plunger, causing the plunger to advance in the direction of arrow A. This causes a blade, such as a razor blade or cutting blade, to press the sling against a distal portion of the cutter, the pinch point, severing the sling. In one example, the surgeon implants a STRATASIS® TF sling, available from Cook Urological Incorporated, Spencer, Ind., under a urethra of a female patient, and draws the ends of the sling through the abdomen of the patient. The cutter herein described is then used to trim the ends of the sling about 3 mm above the rectus fascia, on both sides of the sling.

The blades used in embodiments described herein are flat in the sense of lying generally in a single, Cartesian plane, i.e., they are not curved. The term flat, in this use, includes blades that are tapered in the longitudinal direction, i.e., blades that become thinner as the cutting edge is approached, and also includes blades that are tapered across the width of the blade, i.e., a guillotine-style blade. The term is also meant to include blades with minor variations in thickness, i.e., blades whose edges are serrated. A flat blade includes all these types of blades. Of course, the wedge used with the flat blade may have a corresponding shape also, i.e., completely flat and planar, tapered across the width of the cut, and so on.

The surgical cutter may be used in other ways. For instance, the cutter may be advanced into the patient along a percutaneous tract for cutting an object, such as a suture, inside a patient. In one example, a surgeon performing a percutaneous nephrostomy may require sutures in the tract or within the kidney itself. Trimming the suture and leaving as little foreign matter as possible within the patient may minimize the chances for infection. The cutter may also be used endoscopically, with a surgeon advancing the cutter through the working channel of an endoscope in order to cut or trim an object inside an patient.

The housing may take on many forms, one of which was shown in FIG. 1, and whose details are depicted in FIGS. 1a and 1b. In this embodiment, the housing is inexpensively molded in two halves, top and bottom, which are then snap-fit together. Each half has a handle portion, one handle on one side of the housing and the other handle on the other side, each handle meant to be gripped by a finger of the user. FIG. 1a depicts bottom half 12b with right handle 16b, while FIG. 1b depicts top half 12a with left handle 16a. In this embodiment, any reasonably rigid, medically-acceptable plastic may be used, including ABS and acetal. Other embodiments may use more flexible plastics or elastomers. Rigid means that the housing will not deflect while the surgeon is maneuvering the housing into the patient, and that the housing will not deflect while a cutting operation is being performed.

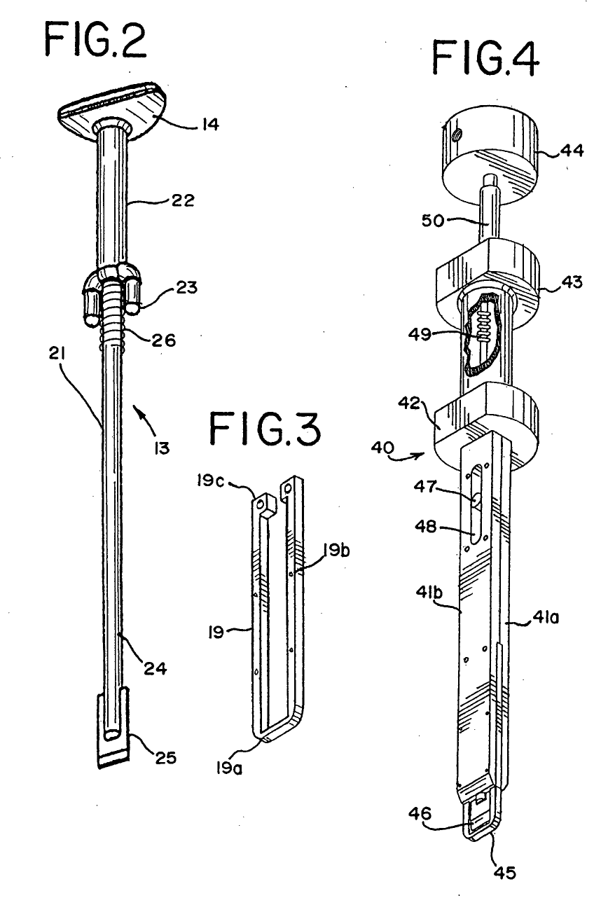

The housing halves may include a number of features, as shown in FIGS. 1a and 1b. The housing halves may include male snap-fit features 12c along their lengths, with mating female portions 12d, so that the lengths of housing halves 12a, 12b may be snapped together. Male snap-fit features 12c and mating female snap-fit features 12d make up snap-fit features to hold together the length of the housing. The housing halves include a space 12j for operating portions of the cutter, a spring and its mount, as seen in FIG. 2, and a lip 12k, to react the spring.

The upper portions of housing halves 12a, 12b may also have snap-fit connections, as shown with male snap-fit feature 12f and female snap-fit space 12h. Each half, 12a, 12b, may have a male and a female snap-fit feature, so that the halves are held together by two snap-fit connections. Alternatively, or in addition, the halves may include bosses 12g, to be used if the halves are to be secured to each other with fasteners, such as small screws or bolts.

More details of plunger 13 are seen in FIG. 2. The plunger includes a shaft 21, an actuation surface or button 14 on its proximal end 22, which may be thicker or thinner than shaft 21. At the distal end 24 of the plunger, a blade 25 may be mounted for trimming the sling, as described above. Plunger 13 also includes a mount 23 for a spring 26, which urges the plunger and blade away from the pinch point at the far distal end of the cutter. The distal portion of the blade itself is referred to as a cutting surface. The opposite surface, against which the sling or other material to be cut is pressed, is referred to as a pinch point. Spring 26 and mount 23 are placed so that the spring is within pocket 12j within housing portions 12a, 12b, and reacts against a retaining feature, lip 12k, when the plunger is actuated by depressing button 14.

Other features within the housing halves may also be used to retain and react the springs. The spaces 12e between the snap-fit features may be used for springs. The male snap-fit features may protrude sufficiently into spaces 12e so that they retain and react one end of the springs when the plunger is depressed by a user. In other embodiments, a spring may be placed in space 12e when housing portions 12a, 12b are assembled.

When the cutter is not in use, the spring urges blade 25 away from the distal end of the cutter. If the cutter is sufficiently long, blade 25 or its distal cutting surface may not be visible in opening 17. After a surgeon or other medical professional has used the cutter, the spring urges the plunger and blade away from the distal end and pinch point, allowing the implanted sling to remain in position within the patient and allowing the severed portion to be removed. The cutter must be able to enter and exit the body of the patient safely, with no danger of cutting in a manner not intended by the surgeon or other user of the device. The blade is preferably masked or hidden by the housing when not actually being used for cutting.

Cutter 10 may use the inner portion of distal end 18 of housing 12 as a pinch point or end stop for the blade. If the plastic is sufficiently rigid, end 18 will suffice as a pinch point. Alternatively, FIG. 3 depicts an end-stop 19, made of steel or other hard material, which may be mounted on the distal end 18 of cutter 10 as part of housing 12. End stop 19 is mounted with closed end 19a at the far distal end of the cutter, and open end 19c mounted in a more proximal position. The inner surface of closed end 19a forms a pinch point when blade 25 urges a urethral sling or other device against the inner surface, severing the sling or other device. End stop 19 may be insert molded into housing 12 as part of the housing, or may be fastened using one or more fasteners (not shown) and one or more apertures 19b in end stop 19. End stop 19 also acts as a guide and an edge for distal end 18 of the housing, forcing the width of the sling or other object being severed to come in contact with the pinch point or the blade, and preventing any portion, such as the sides of the sling or other object, from not coming in contact with the blade and thus being severed. Alternatively, blade 25 may ride in a groove inside end stop 19 or distal end 18, to insure that blade 25 comes into contact with all portions of the sling or other object being severed. This helps to ensure that the blade 25 remains within the bounds of the cutter and the housing, and does not extend, for example, outside the housing or outside the end-stop, i.e., the blade does not extend outside the cutter during any part of a cutting operation.

The cutter may be useful in a number of ways besides trimming slings. For instance, certain procedures require a surgeon to suture a patient. While the sutures must remain in place to be effective, it is desirable to minimize the amount (length) of suture that remains. The cutter may be used to trim the suture, the surgeon subsequently removing as much of the suture as possible from the patient. The cutter is preferably used endoscopically or percutaneously to access and trim the suture, but may also be used in open surgery in general cutting service.

It is also possible to use the cutter to remove body tissue, such as tissue for a biopsy or tissue that is desired to be excised from a patient. Graspers or manipulators may be used to position the tissue for cutting as described above by the cutter embodiments. It is desirable to excise such tissue while taking steps to prevent it from touching other body parts, such as by enclosing the cut tissue in a laparoscopy bag or in an enclosed container, such as the jaws of a biopsy forceps. In one example, a three-prong or four-prong grasper may be passed through the space of a cutter between the blade and the distal end. The grasper is then used to bring the desired tissue or sample into the space for cutting. While still grasping the severed part, it is preferably placed into a container or bag for removal from the patient. The cutter allows for a very safe cutting operation within the patient.

Other embodiments of the cutter may also be used. For example, FIGS. 4 and 5 depict a cutter 40 and plunger 50 with a different configuration. Cutter 40 includes a body formed from upper and lower body portions 41a, 41b, spaced-apart finger grips 42, 43, and an end-stop 45 that is assembled to upper and lower body portions 41a, 41b. Body portion 41b has a slot 48 whose purpose is explained below. The cutter includes plunger 50 with actuation surface or button 44. Spring 49 is captured between plunger 50 and body portions 41a, 41b, urging the plunger in a proximal direction.

Plunger 50 includes a shaft with a distal portion 51 and a proximal portion 52, which may have different diameters. Actuation button 44 may be mounted to proximal portion 52 with a fastener, such as a set screw in the orifice shown, or may simply be mounted via an interference fit or snap fit after other portions of cutter 40 are assembled. The plunger may also include a lip 55 for retaining spring 49. Spring 49 is retained proximally by lip 55 and distally by a similar lip or other retaining surface on the insides of housing portions 41a, 41b, for reacting the spring. The plunger may also include orifice 53 for mounting a transverse pin 47. Pin 47 rides in the slot 48 shown in housing top portion 41b. The distal end of the cutter, with the blade, may be well inside the patient and not visible to the surgeon. The pin, visible through the slot in the side of the cutter body, allows the surgeon or other medical professional using the cutter to gauge the travel of the plunger and thus the blade during the cutting procedure. Housing bottom portion 41a may have a similar slot oppositely positioned for the opposite side of pin 47.

The end 45 or part or all of the blade 46 may be radiopaque or echogenic, or other portions of the cutter may have radiopaque or echogenic portions, in order to enhance the use of visualization techniques. These techniques may include fluoroscopy, x-rays, or ultrasound techniques.

Plunger 50 also has a blade 46 or cutting tool attached to its distal end. The blade may be custom made and laser welded or secured in any satisfactory manner to the distal end of the plunger. Alternatively, the plunger may be made in such a way that allows assembly of the blade into the distal end of the plunger. For instance, as shown in FIG. 8, blade 57 may be made with a retaining opening 58 that allows blade 57 to be insert molded into plunger 56. The blade is positively retained in the plunger without the need for complicated and difficult machining of space 56a, which is occupied by the blade after molding. Alternatively, the plunger may be molded and machined, and blade 57 inserted and retained, such as by using an adhesive or otherwise securing the blade in place. Insert molding, welding, brazing, and other permanent techniques are referred to as methods for integrally attaching one part to another, as opposed to other techniques, such as using fasteners, which may be readily removed.

The embodiments described above generally orient the cutting surface of the blade perpendicularly to the width of the sling, assuring a clean cut transverse to the length of the sling. The distal end of the cutter may also be angled, preferably so that the length of the sling remains perpendicular to the blade, but not the width. FIG. 6 depicts an embodiment of a cutter in which the housing 61 has a slot 62 which is angled with respect to the width of a sling 64 that is drawn through an opening at the distal end 63. Sling 64 remains oriented roughly perpendicularly along its longitudinal axis to the longitudinal axis of housing 61, the direction of arrow B. Sling 64 is drawn through the opening via suture 65 and is then drawn into the slot 62. As sling 64 is drawn into slot 62, and a blade is advanced distally, along the direction of arrow B, the distal portion of slot 62 acts as an opposing surface or pinch point, and the sling is cut as the blade advances. In this embodiment, the blade does not contact the entire width of the sling in a perpendicular manner, but rather at the angle which slot 62 presents to the longitudinal axis of housing 61, angle C.

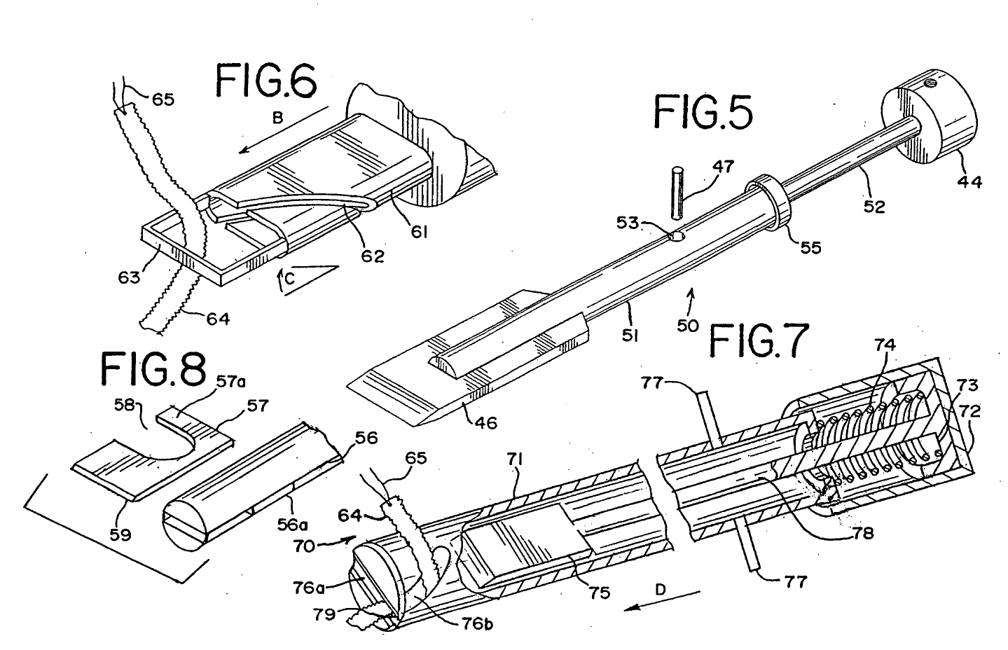

Another embodiment may use a blade that is stationary while the sling is captured by the housing and then brought into contact with the blade. FIG. 7 depicts a cutter 70 that includes a stationary housing portion 71 and a movable portion 72. In this embodiment, stationary portion 71 and movable portion 72 function as a housing for cutter 70. Movable portion 72 mounts a moving wedge 75 connected by a shaft 78. Stationary portion 71 includes a stationary blade 79 inside the distal end of stationary housing 71. Stationary housing portion 71 also includes a first slot 76a on the distal end of portion 71, and a second slot 76b on a top surface of portion 71, second slot 76b angled with respect to the width of blade 79 but perpendicular to the length of the blade.

When a surgeon desires to trim sling 64, the surgeon threads suture 65 through slot 76a and a slot 76b in stationary portion 71, bringing sling 64 into housing portion 71. When the sling is positioned for the desired cut, the surgeon grips stationary portion 71 by handles 77, urging forward movable portion 72 and wedge 75 in a distal direction, the direction of arrow D, bringing wedge 75 into contact with sling 64 and fixed blade 79, cutting the sling. The motion is opposed by spring 74 riding in a space 73 inside of movable portion 72, the spring reacting between the inner portion of movable portion 72 and a lip 78 in the proximal portion of housing 71. After the cut is made, the spring urges housing 72 back, in the direction opposite to arrow D.

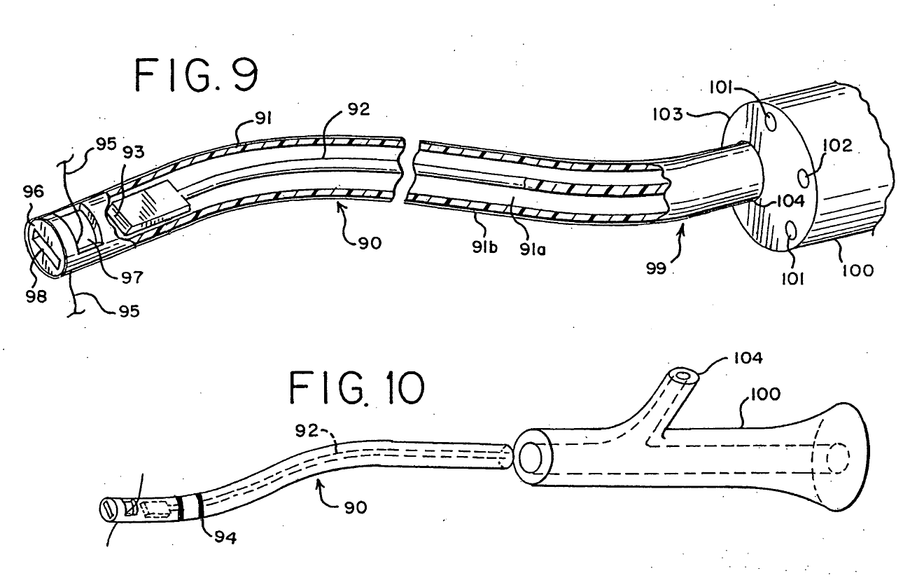

Cutter embodiments may be relatively rigid or they may be relatively flexible. For instance, a cutter may be used endoscopically, if it is sufficiently flexible to travel through an access sheath or an endoscope for use outside the distal end of the endoscope. FIGS. 9 and 10 depict an embodiment of a flexible cutter. Cutter 90 may be used in conjunction with the working channel 104 of an endoscope 100. The distal end 103 of endoscope 100 may also include a lens 102 and one or more light sources 101 so that a surgeon or other medical professional may manipulate the endoscope and the cutter within the patient.

Cutter 90 preferably includes a flexible sheath 91, which may be made from polyimide, polyethylene, polypropylene, urethane, Thoralon® (urethane/siloxane blend), or other flexible, medically-acceptable plastic or elastomeric material. Sheath 91 may include fiber or wire reinforcement in order to maintain its shape and its central lumen 91a. The reinforcement should be sufficient to maintain or regain the lumen, but not so that the sheath looses the flexibility necessary to be useful in endoscopic applications.

The sheath may also have a thin coating 91b, which may be a hydrophilic coating, a fluoropolymer coating, or other lubricious coating. The flexible sheath also preferably includes openings 97 along its length, the openings preferably on opposite sides of the sheath and preferably each about 120° wide. Openings 97 provides a way to introduce an object to be cut, such as suture 95, into the cutter. The cutter also preferably includes a wedge 98 inside sheath 91 at the distal end 96 of the sheath. The wedge is contained within the sheath, the wedge facing proximally. The cutter includes a control rod 92, shown attached to a blade 93.

When a user desires to cut suture 95, the suture is threaded through openings 97, so that a portion of suture 95 is between wedge 98 and blade 93. The surgeon or other medical professional then retracts sheath 91 and wedge 98, or advances control rod 92 and blade 93, capturing suture 95 between the blade and the wedge, and severing the suture or other object which is to be cut. Alternately, the wedge may be attached to the control rod, and the blade mounted within the sheath, at its distal end. A handle may be used to control the cutter.

In order to assist in control and utilization of the cutter, there may be one or more bands 94 placed on the sheath 94 or other portion of the cutter, such as on the blade, the wedge, or other housing embodiments. There may be a single band 94 or there may be a plurality of bands, such as the two bands shown in FIG. 10. These bands are preferably radiopaque or echogenic, so that they may be seen by visualization techniques used by the surgeon in employing the cutter.

For instance, if bands 94 are radiopaque, they may include one or more metals known to be radiopaque, such as tungsten, platinum, barium, iridium, palladium, and the like. The bands need not be made of solid metal, but may include, for instance, compounds of a radiopaque metal in a plastic compound. All that is necessary is that the bands be readily visible under a convenient visualization technique. If the bands are echogenic, so that they are better seen with ultrasonic equipment, the bands may be made of stainless steel and may include a plurality of small bumps or depressions on their surfaces.

Other techniques besides the bands may also be used. For instance, in the first embodiment, end-stop 19 could be made from a radiopaque or echogenic in order to guide a user of the cutter. Any other blades, preferably made from metal, could also be made radiopaque by including a radiopaque marker or an echogenic surface to assist the surgeon or other medical professional in guiding and operating the cutter.

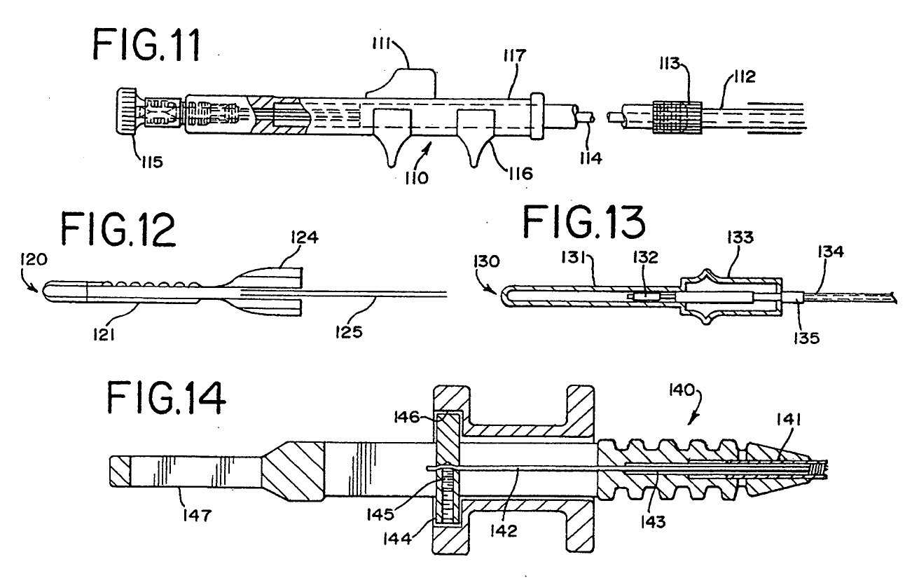

A variety of handles or control devices may be used with embodiments of the cutters of the present invention. Handles that may be suitable include those depicted in FIGS. 11-14. FIG. 11 depicts one suitable handle or control device 110. A sheath 112 and control rod 114 from an endoscopic cutter are attached to the control device 110. In this instance, sheath 112 is fixed to an attachment 113 at a distal end of the control device, while control rod 114 is attached to a movable button 111 for movement by a user. The control rod may be tightened or loosened by knob 115 controlling an internal collet (not shown) or other attachment device. The user controls the device by gripping the handle with the body 117 and fixed finger handles 116 and manipulating control button 111. In other embodiments, the control rod may be fixed and the sheath attached to the collet and movable button. The handles may be used with an access sheath rather than an endoscope.

FIG. 12 depicts a pin vise 120, typically used to control only one of the control rod and sheath. Pin vise 120 may comprise only a handle 121 at its proximal end and a collet 124 at its distal end, used to attach a control element 125 from an endoscopic cutter. The control element may be a fixed or a movable control rod, or may be a fixed or a movable sheath. Two pin vises may be used, one for a control rod and one for a sheath, the control rod at least partially contained within the sheath.

FIG. 13 depicts another handle or control device 130 for controlling both a fixed and a movable control element. Handle 130 includes a body 131 at a proximal end, a movable control button 133 and a distal end 135. In this embodiment, a sheath 134 from a cutter is fixedly attached to the distal end 135, and a control rod 132 is attached to the movable control button 133. A user controls the cutter by manipulating control button 133 back and forth to extend or retract control rod 132. In other embodiments, the sheath may be attached to the movable button and the control rod fixed to the distal end of the handle.

A thumb-ring type handle 140 is depicted in FIG. 14. The handle attaches a sheath 141 fixedly and a control rod 142 movably. Control rod 142 is attached to a cross piece 144 by means of a set screw 145. The cross-piece is attached to a movable finger-spool 146. A user holds the handle securely with thumb-ring 147 and controls the cutter by manipulating movable finger-spool 146 back and forth, extending or retracting control rod 142 and thus operating the cutter. In some embodiments, the sheath may be movable and the control rod fixed to the distal end of the handle. Any of the handles of FIGS. 11-14 may be used with flexible cutters, as with an endoscope, or with relatively rigid cutters, such as those which may be preferred for operations involving percutaneous procedures.

The cutter has been described primarily with respect to trimming urethral slings. The cutter may have many other uses as well. Repairs to the pelvic floor area may include slings or materials, such as vaginal wall slings, used to repair rectocele, enterocele, perineocele, or other defects, such as vaginal vault prolapse. Any materials used in procedures to repair these areas may be trimmed using the cutter. In addition, slings or lengths of materials are used in other areas of the body, such as for repair of hernias.

It is therefore intended that the foregoing detailed description be regarded as illustrative rather than limiting, and that it be understood that it is the following claims, including all equivalents, that are intended to define the spirit and scope of this invention.

What is claimed is:

1. A cutter for cutting an object inside a body, the cutter comprising:
a housing having a longitudinal axis;
a plunger mounted within the housing and configured for longitudinal, manual actuation, a distal end of the plunger mounting one of a flat blade and a wedge, and a distal end of the housing fixedly mounting the other of the flat blade and the wedge; and
an opening in the housing between the flat blade and the wedge, the opening configured to admit an object for cutting and not to admit a part of the body, and wherein the blade is contained within the cutter.

2. The cutter of claim 1, further comprising a handle mounted to the housing.

3. The cutter of claim 1, wherein the plunger is mounted to the housing with a spring.

4. The cutter of claim 1, wherein one of the flat blade and the wedge is integrally attached to the distal end of the plunger, or the other of the flat blade and the wedge is integrally attached to the distal end of the housing.

5. The cutter of claim 1, wherein the housing is a flexible sheath and further comprising a handle mounted remotely from the housing.

6. The cutter of claim 1, further comprising a pin, wherein the housing further comprises a slot, the pin is secured to the plunger and rides in the slot.

7. The cutter of claim 1, further comprising at least one feature selected from the group consisting of radiopaque and echogenic.

8. The cutter of claim 1, wherein the housing comprises a flexible tube with an opening between the tube and a distal end of the flexible tube, wherein the distal end of the tube is stiffer than a length of the tube.

9. The cutter of claim 1, wherein the housing further comprises a lubricious coating.

10. A cutter for cutting an object inside a body, the cutter comprising:
a rigid housing;
a handle operably mounted to the housing;
a plunger mounted inside the housing and mounting one of a flat blade and a wedge, the plunger configured for longitudinal, manual actuation, wherein the other of the flat blade and the wedge is fixedly mounted to the housing; and
a first slot in the housing oriented at an angle to the housing.

11. The cutter of claim 10, wherein the plunger is movably mounted to the housing with a spring, the plunger includes the wedge at a distal end of the plunger, and the housing further comprises the flat blade at a distal end of the housing.

12. The cutter of claim 10, further comprising a second slot in a distal end of the housing.

13. The cutter of claim 10, wherein the housing comprises two portions, a first housing portion mounted fixedly to the plunger, and a second housing portion mounted to the first housing portion via a spring.

14. The cutter of claim 13, wherein the plunger mounts the blade and the second housing portion further comprises the wedge.

15. A method of cutting an object inside a body, the method comprising:
placing a cutter into the body of a patient, wherein the cutter comprises a housing having a longitudinal axis and a distal opening, a plunger mounted within the housing for manual, longitudinal actuation, the plunger mounting one of a flat blade and a wedge, and a distal portion of the housing fixedly mounting the other of the flat blade and the wedge;
drawing the object into the opening;
pushing on a portion of the cutter to bring a cutting surface of the flat blade into contact with the object to cut the object; and
removing the object which was cut.

16. The method of claim 15, wherein the plunger is fixed, and the step of pushing on the cutter comprises drawing the object across an edge of the blade exposed in the opening.

17. The method of claim 15, wherein the plunger is movable, and the step of pushing on the cutter comprises depressing the plunger.

18. The method of claim 15, wherein the object is drawn into the opening by pulling a suture attached to the object into the opening.

19. The method of claim 15, wherein the cutter is adapted for placement within the human body, and in which a cutting surface of the blade does not contact the body.

20. The method of claim 15, further comprising guiding the cutter by an x-ray or ultrasonic visualization technique.
